(19) United States
(12) Patent Application Publication
(10) Pub. No.: US 2006/0178554 A1
(43) Pub. Date: Aug. 10, 2006

(54) Nerve protection barrier

(76) Inventor: Shlomo Sam Mandel, Bloomfield Hills, MI (US)

Correspondence Address: Shlomo Mandel, 4554 Walden Dr., Bloomfield Hills, MI, 48301, US

(21) Appl. No.: 11/050,171

(22) Filed: Feb. 4, 2005

(51) Int. Cl. A61F 2/02

(52) U.S. Cl. 600/37; 623/23.72

(57) ABSTRACT

A nerve protection barrier having a compression resistant covering which surrounds and protects a portion of the nerve from impingement by surrounding tissues. The compression resistant covering has a radial dimension which is larger than the outer diameter of the nerve, and an axial dimension which is long enough to protect at least a portion of the nerve which is at risk of impingement by surrounding tissues. The compression resistant covering being resistant to compression in the radial dimension, while being flexible in the axial dimension and is formed from one or more bio-compatible materials selected from the group consisting of polymers, metals, alloys, ceramics and composites thereof. The compression resistant covering may also protect the nerve from the surrounding chemical environment. The nerve protection barrier may further contain time released therapeutic drugs/medications.

### FIELD OF THE INVENTION

The present invention relates generally to medical devices and methods of their use and more specifically to medical devices for the mechanical and chemical protection of nerves which have become irritated and painful due to compression.

### BACKGROUND OF THE INVENTION

Back pain will strike nearly every one of us at some time or another. Indeed, statistics in this country show about an 80% lifetime incidence. When the degree of discomfort and related limitation of function is considered, the enormous magnitude of the problem can be fully appreciated. From an economic perspective, current estimates put the cost of direct medical care and indemnity benefits at nearly 150 Billion dollars per year.

The effort to reduce the burden and ease the suffering is equally impressive. Practitioners from various backgrounds, using all kinds of medications, instruments, and manual treatments profess to have the answer to an aching back. The main stream of allopathic medicine has followed a trend, which is increasingly dominated by more invasive and expensive operations. Yet, there is no demonstrable improvement in outcomes, while the cost continues to escalate. Finding a technique, which can achieve better results, with lower morbidity and cost, would be a significant discovery.

Why the back hurts, and where the pain comes from are naturally important questions for which there are no clear answers. In spite of tremendous diagnostic capability gains, where MRI today can safely and delicately image the spine, the source(s) of the pain remain largely illusive. However, scientific studies have successfully narrowed the potential origin of pain in the spine. One cause, if not the principle cause of pain, is the spinal nerve root. Recent investigations show that it is sensitive to mechanical compression by bone or disc material.

When the path of the root is blocked, pain is produced, and nerve transmission is impaired. In other words, pain in the lower back or leg, accompanied by numbness and weakness is the typical clinical presentation of a compressed spinal nerve root. Recently it was discovered that pressure on the root actually produces a chemical reaction wherein inflammatory substances are secreted. This is a likely cause of the pain sensation. Therefore, much of the effort to treat back pain has been directed at protecting the nerve root. However, to date, an effective, safe, and long lasting method to treat neurogenic pain has not been found.

### SUMMARY OF THE INVENTION

The present invention is a nerve protection barrier. The basic form is a compression resistant covering which surrounds and protects a portion of the nerve from impingement by surrounding tissues and may also protect the nerve from the surrounding chemical environment. The compression resistant covering has a radial dimension which is larger than the outer diameter of the nerve, and an axial dimension which is long enough to protect at least a portion of the nerve which is at risk of impingement by surrounding tissues. The compression resistant covering being resistant to compression in the radial dimension, while being flexible in the axial dimension and is formed from one or more bio-compatible materials selected from the group consisting of polymers, metals, alloys, ceramics and composites thereof.

The most basic embodiment of the nerve protection barrier of the instant invention comprises a tube-like shape of varying radial diameter to accommodate the varying diameter of the nerve it protects. Another embodiment comprises a tubular clip shape having a longitudinal slot in the wall thereof. Yet another embodiment of the nerve protection barrier of the present invention comprises a coil or spring structure. A fourth embodiment comprises a clam shell structure, having at least two portions which fit together to cover and protect the nerve. The clam shell structure may have mating tabs which either connect together to lock the barrier in place around the nerve or are used to lock the nerve protection barrier to the surrounding bone. An additional embodiment of nerve protection barrier of the present invention comprises a blanket of pliable material which is wrapped around the nerve and hardens in-situ.

The nerve protection barrier may be formed from one or more bio-compatible polymers selected from the group consisting of polyurethanes, silicones, polyesters, polycarbonates, polyethylene, polyvinyl chloride, polypropylene, methylacrylate and mixtures or alloys thereof. Alternatively, the nerve protection barrier may be formed from one or more bio-compatible metals selected from the group consisting of titanium, titanium-nickel alloys such as NiTinol (shape memory Ni—Ti metal alloy), cobalt-chromium alloys and 316 L stainless steel. Further materials of construction may include one or more bio-compatible ceramics selected from the group consisting of Al₂O₃ and ZrO₂ and one or more bio-compatible composite materials selected from the group consisting of silicone composites, ceramic composites and thermoplastic-fiber composites.

The nerve protection barrier may further include time released therapeutic drugs/medications embedded therein. Examples of such embedded time released therapeutic drugs/medications are anti-convulsants, sedatives, psychic energizer, drugs affecting osteoblast and osteoclast activity, prostaglandin and substance P inhibitors, glycoproteins, mucopolysaccharides, non-steroidal anti-inflammatory drugs, anti inflammatory cytokines such as IL4, IL6 and IL10, anti-tumor necrosis factor—alpha inhibitor, leukotrien inhibitors and corticosteroids. The embedded time released therapeutic drugs/medications may be embedded in a separate layer from said compression resistant covering.

### BRIEF DESCRIPTION OF THE FIGURES

FIG. 1 is a diagrammatic depiction of a cross section of a normal spine;

FIG. 2 is a diagrammatic depiction of a cross section of a spine in which the intervertebral disc has become herniated or is bulging;

FIG. 3 is a diagrammatic depiction of a cross section of a spine in which part of the bulge of the intervertebral disc which was pressing on nerve root has been resected and the nerve protector of the present invention has been placed around the nerve root to protect it from further compression and irritation;

FIG. 4 which is depiction of a basic embodiment of the nerve protector of the present invention;

FIG. 5a is a cross section of a basic embodiment of the nerve root protector of the present invention;

FIG. 5b is a cross section of another basic embodiment of the instant invention which is impregnated with timed release therapeutic drugs/medications;

FIGS. 6a and 6b depict a coil or spring like embodiment of the nerve protection barrier of the present invention;

FIGS. 7a and 7b depict a clip like embodiment of the nerve protection barrier of the present invention;

FIG. 8 depicts a blanket type embodiment of the nerve protection barrier of the present invention;

FIG. 9 depicts a clam shell type embodiment of the nerve protection barrier of the present invention; and

FIG. 10 is a schematic depiction of a percutaneous type of surgery to place the nerve protector of the instant invention around the affected nerve.

### DETAILED DESCRIPTION OF THE INVENTION

Several parts of the spine are known to be the primary generators of pain. One in particular, is the nerve root, which exists the spinal canal through the neural foramen. The root is a delicate structure, which is frequently compressed and irritated by protruding discs, arthritic facet joints, canal osteophytes and more. Traditional treatment approaches call for resection of offending tissue obstructing the root's path through the canal.

The invention introduced here takes a new and novel approach. Instead of relying on extensive surgery, and its inherent morbidity, the nerve protection barrier or nerve barrier barrier offers a safer alternative for mechanical and chemical protection of the spinal nerve root. The barrier provides biomechanical and neurochemical protection which can be used in conjunction with traditional or minimally invasive surgery.

Current scientific data supports the thesis that most neurogenic back pain comes from a combination of mechanical and chemical irritation of the spinal nerve root. Recent studies have further quantified the threshold of compression required to produce sciatic pain. Likewise, the identity of substances which mediate the chemical inflammation are also known. The advances in material fabrication, which facilitate the construction of protection barriers, coupled with operative techniques for safe and effective implantation, pave the way for the development of the new nerve protection barrier. Whereas in the past, so called minimally invasive operations were limited by the frequent relapse of compressive lesions and corresponding symptoms, this new device will provide long term barrier protection from various sources of mechanical and chemical challenges.

In support of the expected benefits which the nerve protection barrier brings, is a long and well documented history of successful treatment through removal of external pressure on the nerve, and deployment of anti-inflammatory substances. Doctors have used a myriad of operative techniques to decompress the nerve. In addition, or occasionally in lieu of surgery, medications are used to mitigate the chemical irritation around the nerve. The novelties of the barrier are that, 1) it combines the benefits of both approaches; and 2) the barrier allows for long lasting effect and therefore, pain relief. How well indeed the barrier protects the nerve is the subject of current investigation.

Placement of a physical barrier around the spinal nerve root can provide clinically effective protection from mechanical, biochemical, and electrophysiological compression and irritation. The latest scientific investigations show that the nerve root is the epicenter of neurogenic back pain. Recent studies also demonstrate the pressure threshold and chemical or hormonal affect on the root. In the past, surgical treatment consisted exclusively of cutting or removing objects that obstructed or constricted the root's path. The barrier introduced here would provide an alternative or adjunctive function by engulfing and shielding the root against existing or reoccurring obstacles.

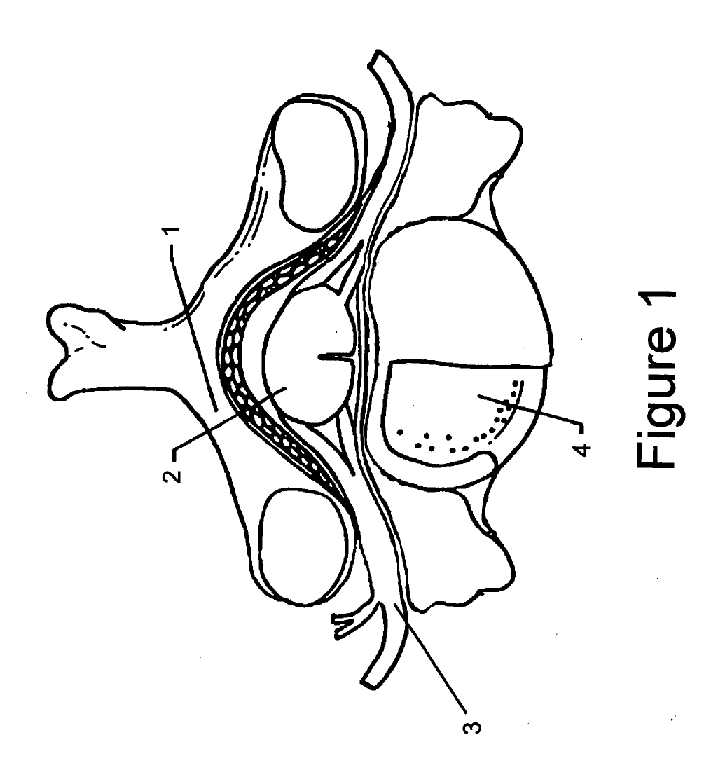

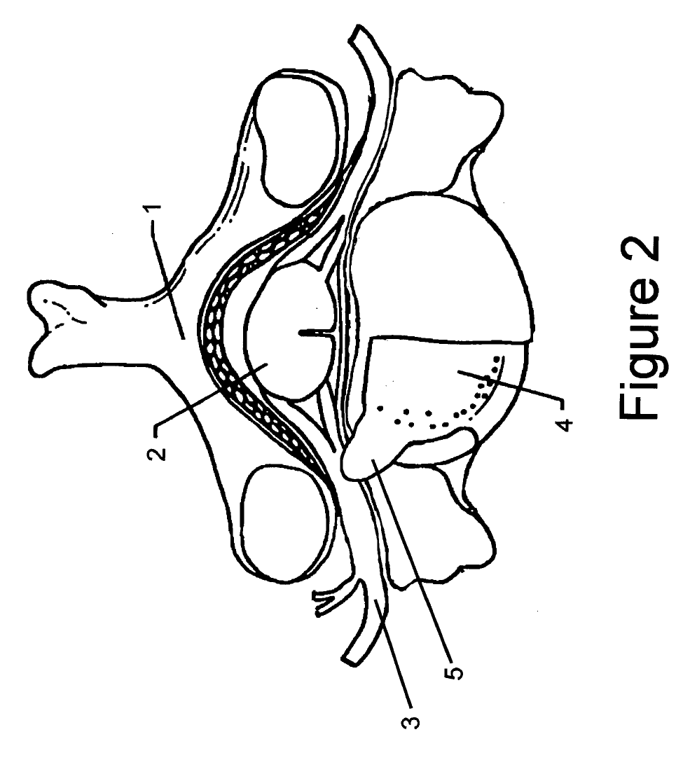

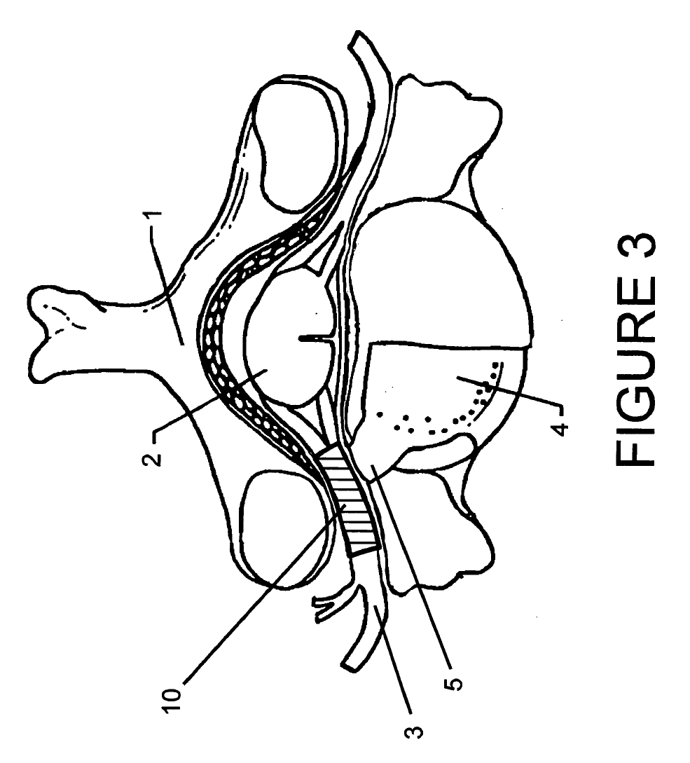

FIG. 1 is a diagrammatic depiction of a cross section of a normal spine. The vertebra 1, spinal cord 2, nerve root 3, and intervertebral disc 4 are all in normal condition. However, FIG. 2 is a diagrammatic depiction of a cross section of a spine in which the intervertebral disc 4 has become herniated or is bulging 5. The bulge 5 is compressing and irritating nerve root 3. FIG. 3 is a diagrammatic depiction of a cross section of a spine in which part of the bulge 5 of the intervertebral disc 4 which was pressing on nerve root 3 has been resected. Additionally, the nerve protector 10 of the present invention has been placed around the nerve root 3 to protect it from further compression and irritation.

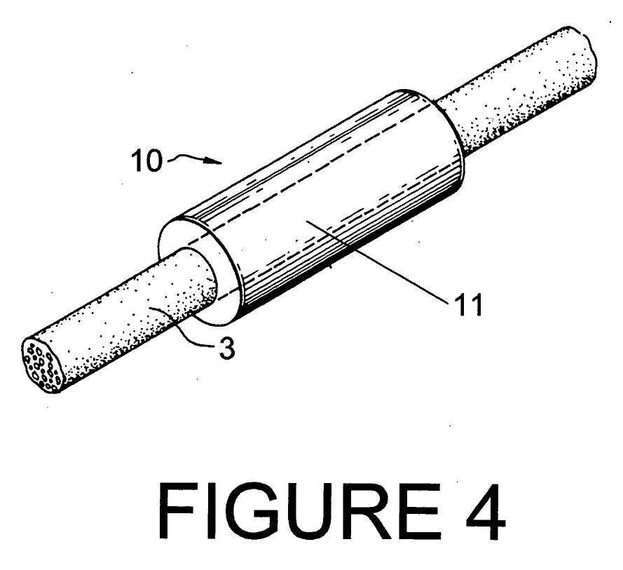

A shown in FIG. 4, which is depiction of a basic embodiment of the present invention, the nerve protector 10 is composed of a tough, strong outer layer or sheath which surrounds and protects the nerve root 3. This outer layer forms a physical barrier that resists compression by the surrounding tissues such as bone, bulging vertebral disc material, etc. Structural integrity of the barrier must provide for protection from compression or distortion of the nerve caused by intervertebral disc material displacement, ligamentum flavum, facet joint arthritis, osteophytes, or postoperative scarring, while allowing for normal flexion, rotation, side bending and extension motion of the spine, as well as freedom of movement for the root itself. That is, the protector's physical properties and location anatomically, will not compromise nerve or tendon movement, nor vertebral body motion. Likewise, the outer shell, while giving rigid protection, will be flexible to accommodate for example the wider dorsal root ganglion.

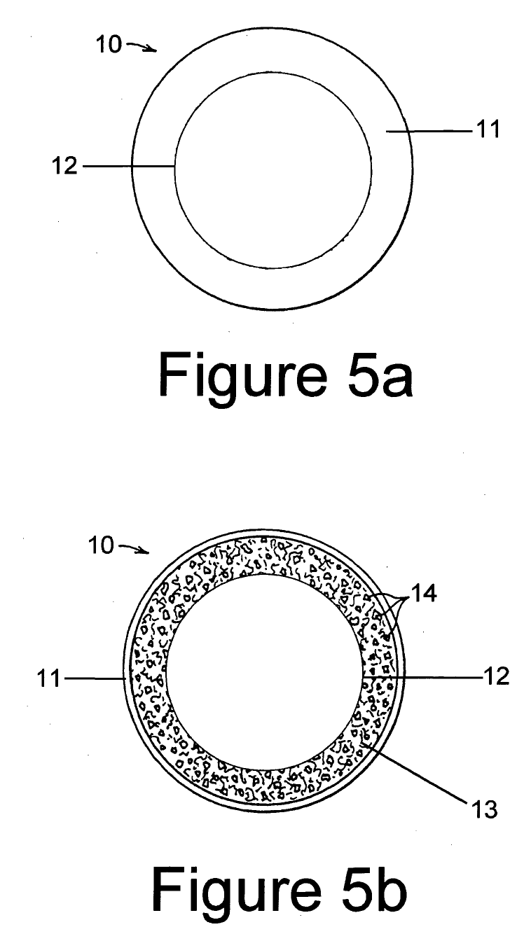

As shown in FIG. 5a, which is a cross section of a basic embodiment of the nerve root protector of the present invention, the outer layer 11 of the nerve protection barrier 10 is, in its most basic of shapes a tube-like structure which surrounds and mechanically protects the nerve. On the inside of the tube-like structure, the inner lining 12 is smooth and allows unencumbered movement of the nerve. Other configurations would be tent like or stent type structures. A flat or slightly curved barrier, anchored against set bone landmarks in the canal may also be used. The outer walls of the barrier may be supported by the bone landmarks of the neural foraminal, or spinal canal.

Substances used to make the barrier include but are not limited to bio-compatible polymers, metals, ceramics and composites thereof. Examples of polymers include polyurethanes, silicones, polyesters, polycarbonates, polyethylene, polyvinyl chloride, polypropylene, methylacrylate. Metals include titanium, titanium-nickel alloys such as NiTinol (shape memory Ni—Ti metal alloy), cobalt-chromium alloys and 316 L stainless steel. Ceramics include Al₂O₃ and ZrO₂. Composites include silicone composites, ceramic composites and thermoplastic-fiber composites. A bio-absorbable substance may be employed in certain applications where a permanent barrier is not indicated.

Size of the barrier will vary. Ideally, the pre-deployment process includes imaging and measurement of the neural foraminal canal. Thus, the size, including diameter and length, along with the configuration will be determined prospectively through sophisticated, noninvasive imaging studies, and will be custom made for each application. Each protector will be fitted prospectively to the dimension and configuration of the patient. Materials used in the construction of the nerve protection barrier should be MRI compatible. The typical barrier will be between 2 mm and 8 cm in length, and will vary in diameter from about 3 mm to 24 mm.

It should be noted that the nerve protector may be deployed in multiple locations or levels of the spine and bilaterally. Also, the nerve protector barrier of the present invention can also be used in areas away from the spine. For example, nerve compression at the wrist, known as carpal tunnel syndrome, and ulnar nerve dysfunction of the elbow known as cubital tunnel syndrome could also be alleviated by the use of the present nerve protector. Yet another use for the such a protective barrier would be protection of the injured and repaired tendons and cases of peroneal tendinitis. If necessary, the nerve protector can be easily removed and the nerve protector causes no adverse affect on local blood supply, or collateral innervation.

In addition to a physical barrier, the protector will create a better chemical environment for the nerve root. Studies show that pain sensation is a function of both extrinsic pressure and exposure to noxious chemicals and hormones. Therefore, as shown in FIG. 5b, which is a cross section of another basic embodiment of the instant invention, the nerve protector 10 may contain an additional layer 13 which is impregnated with timed release therapeutic drugs/medications 14 to overcome the effects of the offending substances. The outer layer 11 is still of rigid, compression resistant construction and the inner lining 12 is still smooth to the nerve. The impregnated drugs/medications 14 include, but are not limited to, anti-convulsants, sedatives, psychic energizer, drugs affecting osteoblast and osteoclast activity, prostaglandin and substance P inhibitors, glycoproteins, mucopolysaccharides, non-steroidal anti-inflammatory drugs, anti inflammatory cytokines such as IL4, IL6 and IL10, anti-tumor necrosis factor—alpha inhibitor, leukotrien inhibitors and corticosteroids. The active agent will be gradually released in the area adjacent to the nerve root. Medication release by the barrier is steady paced, reliable and long term. It represents a significant improvement over the current injection method, which can be haphazard and lasts for only a short period of time. Dose of drugs and rate of release will vary, according to anticipated patient need. Prior to introduction, the optimal dose is determined and packaged.

The physical barrier provides a therapeutic insulated chemical environment. The protector will limit noxious neurochemical exposure of the nerve by physical and chemical means. In addition to structural support, the protector wall will prevent contact of the nerve with locally secreted inflammatory, neurotoxic chemical or hormones.

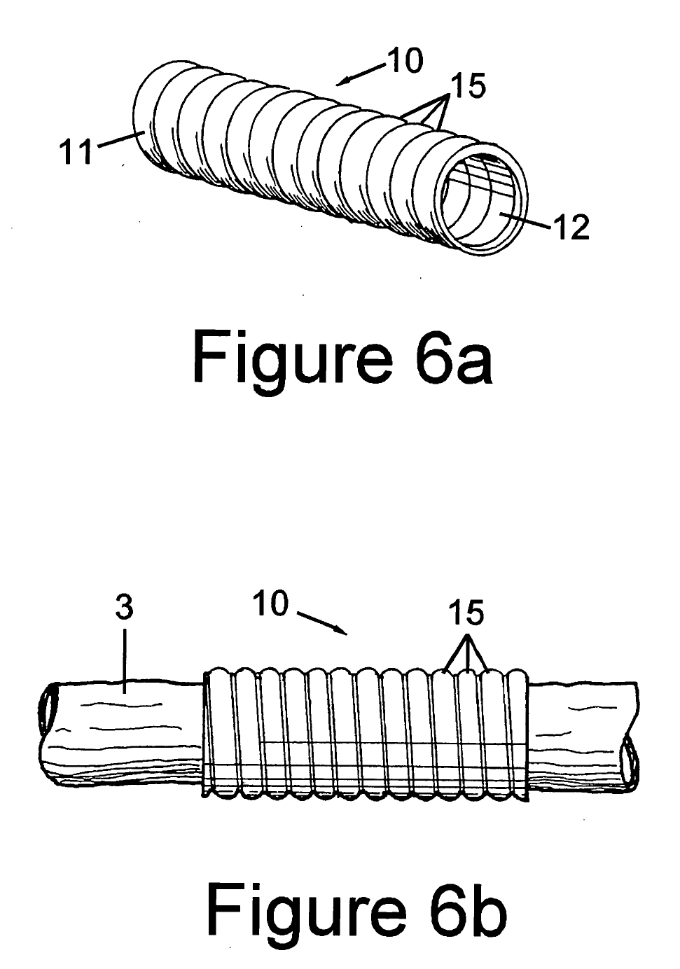

Illustrative examples of a few embodiments of the nerve protector 10 of the present invention are shown in FIGS. 6, 7, 8 and 9. Further discussion of these barriers and their method of placement around the nerve are presented below. FIGS. 6a and 6b depict a coil or spring like nerve protection barrier 10. It consists of multiple coils 15, which make up the compression resistant layer 11. The inner surface 12 is smooth to the nerve 3.

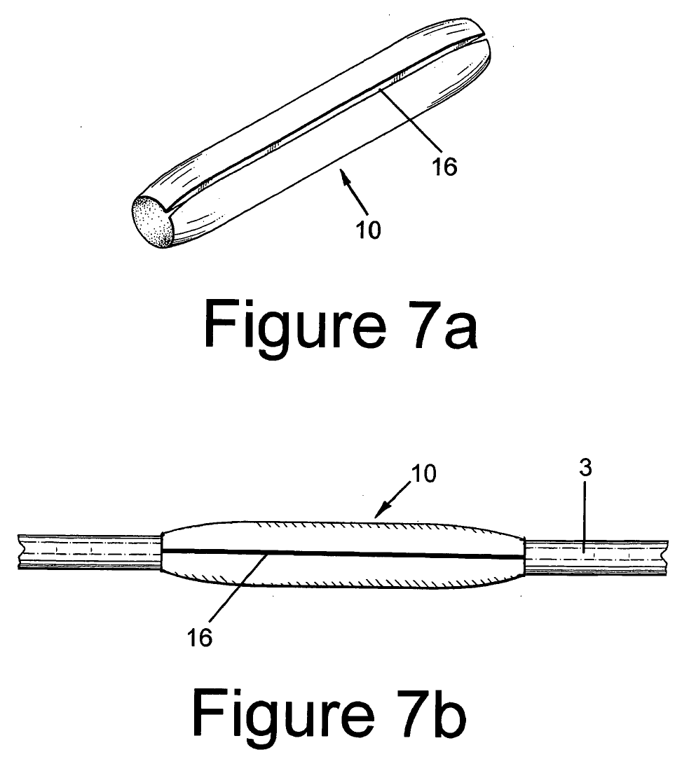

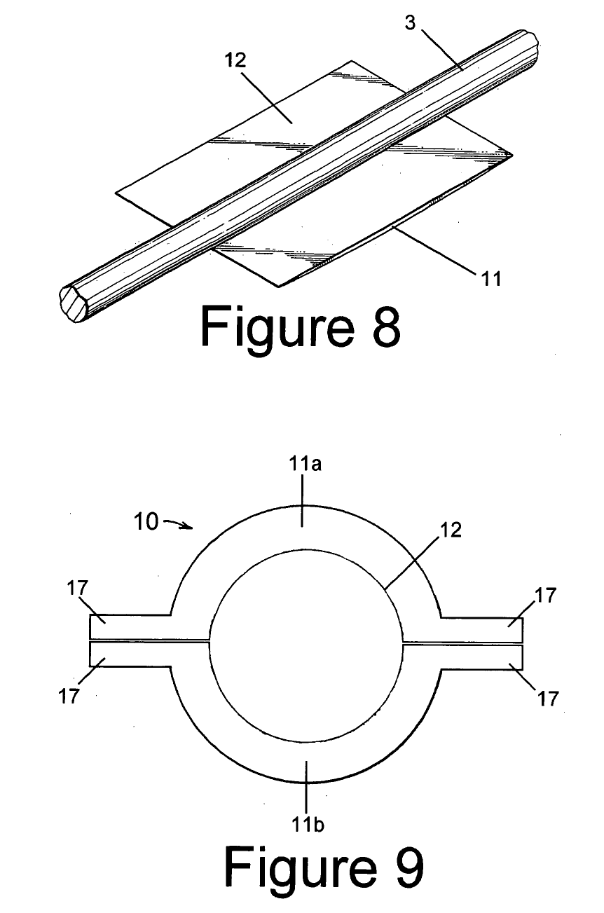

FIGS. 7a and 7b depict a clip like nerve protection barrier 10. It has a slot 16 which can be expanded and then released to place the barrier around the nerve 3. FIG. 8 depicts a blanket type nerve protection barrier in which a initially soft outer layer 11, which has a smooth inner lining 12 is wrapped around the nerve 3. This soft blanket material hardens once in place.

Finally, FIG. 9 depicts a clam shell type nerve protection barrier 10, which is composed of two outer layer halves 11a and 11b. These halves are placed around the nerve such that the smooth inner lining 12 is adjacent the nerve and the mating tabs 17 either connect together or are used to lock the nerve protection barrier 10 to the surrounding bone.

Protector Deployment

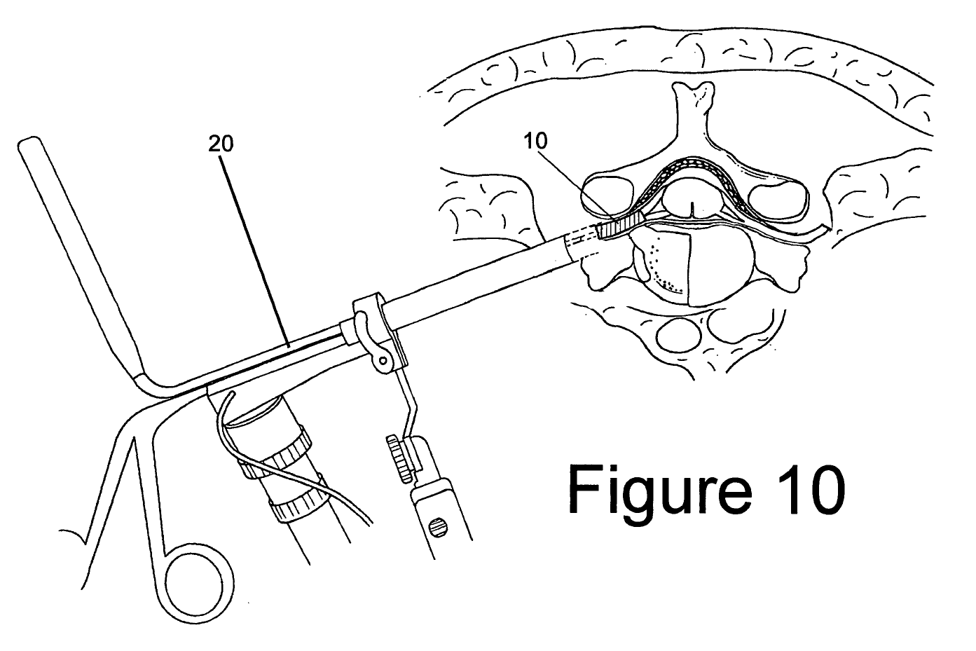

There are a number of surgical methods currently for exposing the canal and spinal nerve root. Broadly speaking, they fall into two main categories: open and percutaneous procedures. The protector is adapted to be used in both instances. For a minimally invasive placement, the launcher is introduced through the working channel of the endoscope. Access to the neural foraminal canal is achieved through a transforaminal technique. It is performed under local anesthesia, and is used with increasing frequency to decompress the nerve root. The barrier presented here would complement percutaneous discectomy by preventing recurrent compression of the nerve root post operatively, which is one of the main drawbacks of percutaneous discectomy. FIG. 10 is a schematic depiction of a percutaneous type of surgery where percutaneous surgical instruments 20 are used to place the nerve protector 10 around the nerve.

One important challenge is finding a method for reliably placing the barrier in position surrounding the nerve root, without injuring the nerve or surrounding structures. To accomplish this, the inventor proposes alternative solutions. First, a ‘clam’ design (like that of FIG. 9), can be delivered by launcher or surgical instruments, and then placed around the nerve in the canal. By placing a guide wire in parallel with the nerve, and deploying the protector over the wire, the risk of injury to the root is minimized.

A second path for open or percutaneous positioning of the protector involves ‘blanketing’ the chosen material around the nerve (as depicted in FIG. 8). The diameter and length of the ‘blanket’ will be determined through preoperative imaging and planning.

A third option for placement of the protector is to coil a spring-like barrier (similar to FIGS. 6a and 6b) around the nerve. All of the suggested techniques share two critical elements. First, each technique allows for positioning of the protector around the root with minimal risk to the nerve itself, and second, the structural integrity of the barrier is maintained, so it may provide for maximum protection.

Patients deriving the most benefit from the barrier will have undergone surgical discectomy, and canal stenosis decompression. Design of the nerve barrier, and launch instruments will allow for introduction in either a traditional open operation, or a transforaminal percutaneous surgery. Selection of the most suitable among the available barriers will depend on the nature of the anatomic problem. Potential options will be explored preoperatively, and the final decision made by the surgeon after the decompression phase is complete. In addition to use of the barrier to protect the decompressed nerve after surgery, it may be deployed prophylactically in cases where findings suggest significant future risk of nerve compression. Application may be indicated at multiple spinal levels or bilaterally. For compression of the ventral thecal sac, a barrier in the form of a band may additionally be deployed, as is known in the art. It will be anchored in place by the anterior aspect of the superior articulating processes. Protection of the dural sac from displaced disc material may be used in conjunction with use of a nerve protector of the present invention in the adjacent canal.

Percutaneous deployment is a minimally invasive, thus carries far less risk of complications, and a faster recovery. Transforaminal access to the canal is well established, and several recent surgeons have described successful decompression of the nerve root, including a discectomy, foraminotomy, and facetectomy.

Reliable and accurate placement of the protector begins with pre operative preparation. Using MRI and CT imaging a graphical representation of the neuro-foraminal canal is performed. The instant inventor has coined the term “neuroforaminogram” to describe this graphical representation. A comprehensive and detailed image is achieved, which will serve as a blue print for the placement procedure.

By applying virtual fluoroscopy (FluoroNav, a registered trademark of Surgical Navigation Technologies, Inc) to the neuroforaminogram data, a pre operative course is chartered. Following the usual skin landmarks, a guide wire is passed through the canal, adjacent to the nerve root. The launcher is then passed over the wire, and positioned in parallel to the nerve. From this point, the protector may be deployed in one of three alternative techniques.

One way of placing a cylindrical object around a continuous structure, is by designing it to function like a clam. Depending on the surgical approach, the clam is deployed by direct or endoscopic visualization. In the percutaneous system, the surgeon will use the endoscope to confirm the preoperative position of the nerve. The launcher will put the protector parallel to the nerve, and the protector barrier is then released by the surgeon. Through the opening of the clam-like barrier, traverses the nerve. After confirming the barrier is safely and correctly positioned, a second pass by the launcher is used to ‘zip’ the open end.

Intra operative monitoring using SSEP and DSEP will be performed during the procedure in order to detect and prevent excessive handling or injury to the nerve while the operation is taking place. To further protect the nerve from iatrogenic injury, the launcher puts a cylinder around the root, and a neuroforamingram with contrast is used to assure proper positioning around the nerve. Only after seeing the root inside the tube, will the barrier be deployed. When the operative field is open, the surgeon may use a similar launcher instrument, but here the nerve is visible, and a neuroforaminogram may not be necessary.

Depending on the size or length of nerve requiring protection, the treating doctor has two available options. First, the barrier is designed to expand, like a spring. So, once the ‘clam’ protector is wrapped around the nerve, the surgeon may pull the leading edge to the predetermined final spot. Second, more than one protector device may be used. They are deployed in series, and their relative location can vary; either detached by desired distance, or touching. Alignment is accomplished by relative positioning of markers on the external surface of the barrier.

Since the nerve diameter is not constant, the barrier size and relative location is designed to conform. The ‘clam’ protector is created with varying diameter and length. Therefore, armed with pre-operative neuroforaminogram data, and direct operator vision, the precise fitting protector can be accurately positioned around variable diameter size segment of nerve. Another method of accommodating a bulge like the dorsal root ganglion is by linking together consecutive segments of the barrier, each one measured to fit the desired nerve position.

Another method of applying the barrier is by coiling a spring-like protector around the nerve. As described earlier, the barrier is deployed after the launcher is in proper position. However, using a piston-like action, or a rotating handle, the coil like barrier is threaded over the nerve. Additional external coating material or medication can be applied to the barrier after its position is confirmed.

The third protector installation technique, is coating or wrapping. New materials recently introduced (specifically NiTinol shape memory alloys) allow for initial application in a soft, moldable form, which hardens once placed in position. From the launcher, a sheet of protector is released and folded in the canal.

Regardless of the deployment method, ultimate configuration of the protector barrier will guard against nerve compression by disc material, bone spurs, hypertrophied ligaments, and inflamed facet joints. The barrier's effectiveness is a function of meticulous positioning, flexible, yet compression resistant materials, and pharmacologically active substances.

The foregoing is provided for purposes of explaining and disclosing some preferred embodiments of the present invention. Modifications and adaptations to the described embodiments, particularly involving changes to the shape, size, design and materials of construction of the nerve protector and its method of placement around the nerve, will be apparent to those skilled in the art. These changes and others may be made without departing from the scope or spirit of the invention in the following claims.

I claim:

1. A nerve protection barrier comprising:
a compression resistant covering which surrounds and protects a portion of the nerve from physical impingement by surrounding tissues;
said compression resistant covering having a radial dimension which is larger than the outer diameter of the nerve, and an axial dimension which is long enough to protect at least a portion of the nerve which is at risk of impingement by surrounding tissues;
said compression resistant covering being resistant to compression in the radial dimension, while being flexible in the axial dimension;
said compression resistant covering being formed from one or more bio-compatible materials selected from the group consisting of polymers, metals, alloys, ceramics and composites thereof.

2. The nerve protection barrier of claim 1, wherein said compression resistant covering further protects the nerve from the surrounding chemical environment.

3. The nerve protection barrier of claim 1, wherein said compression resistant covering comprises a tube-like shape of varying radial diameter to accommodate the varying diameter of the nerve it protects.

4. The nerve protection barrier of claim 1, wherein said compression resistant covering comprises a tubular clip having a longitudinal slot in the wall thereof.

5. The nerve protection barrier of claim 1, wherein said compression resistant covering comprises a coil or spring structure.

6. The nerve protection barrier of claim 1, wherein said compression resistant covering comprises a clam shell structure, having at least two portions which fit together to cover and protect the nerve.

7. The nerve protection barrier of claim 6, wherein said at least two portions of said clam shell structure have mating tabs which either connect together to lock the barrier in place around the nerve or are used to lock the nerve protection barrier to the surrounding bone.

8. The nerve protection barrier of claim 1, wherein said compression resistant covering comprises a blanket of pliable material which is wrapped around the nerve and hardens in-situ.

9. The nerve protection barrier of claim 1, wherein said compression resistant covering is formed from one or more bio-compatible polymer materials selected from the group consisting of polyurethanes, silicones, polyesters, polycarbonates, polyethylene, polyvinyl chloride, polypropylene, methylacrylate and mixtures or alloys thereof.

10. The nerve protection barrier of claim 1, wherein said compression resistant covering is formed from one or more bio-compatible metals selected from the group consisting of titanium, titanium-nickel alloys such as NiTinol (shape memory Ni—Ti metal alloy), cobalt-chromium alloys and 316L stainless steel.

11. The nerve protection barrier of claim 1, wherein said compression resistant covering is formed from one or more bio-compatible ceramics include selected from the group consisting of Al₂O₃ and ZrO₂.

12. The nerve protection barrier of claim 1, wherein said compression resistant covering is formed from one or more bio-compatible composite materials selected from the group consisting of silicone composites, ceramic composites and thermoplastic-fiber composites.

13. The nerve protection barrier of claim 1, wherein said nerve protection barrier further include time released therapeutic drugs/medications embedded therein.

14. The nerve protection barrier of claim 13, wherein said embedded time released therapeutic drugs/medications are selected from the group consisting of anti-convulsants, sedatives, psychic energizer, drugs affecting osteoblast and osteoclast activity, prostaglandin and substance P inhibitors, glycoproteins, mucopolysaccharides, non-steroidal anti-inflammatory drugs, anti inflammatory cytokines such as IL4, IL6 and IL10, anti-tumor necrosis factor—alpha inhibitor, leukotrien inhibitors and corticosteroids

15. The nerve protection barrier of claim 13, wherein said embedded time released therapeutic drugs/medications are embedded in a separate layer from said compression resistant covering.
